(19) United States
(12) Patent Application Publication
(10) Pub. No.: US 2025/0037882 A1
(43) Pub. Date: Jan. 30, 2025

(54) SENSOR FOR DETECTING AN AIRBORNE DISEASE, IN PARTICULAR A RESPIRATORY DISEASE, AND ASSOCIATED SYSTEM AND METHOD

(71) Applicant: ZYNNON AG, Schindellegi (CH)

(72) Inventor: Khaled Abousaleh, Schindellegi (CH)

(21) Appl. No.: 18/721,757

(22) PCT Filed: Nov. 17, 2022
(86) PCT No.: PCT/EP2022/082300

§ 371 Date: Jun. 19, 2024

(30) Foreign Application Priority Data
Dec. 21, 2021 (FR) FR2114141

(51) Int. Cl. G16H 50/80; A61B 5/00; A61B 5/01; A61B 5/08

(52) U.S. Cl. CPC G16H 50/80; A61B 5/0002; A61B 5/01; A61B 5/0823

(57) ABSTRACT

A device for determining an occurrence of an airborne disease, in particular a respiratory infection in an individual, includes: an infrared sensor or camera; a microphone; a sensor for measuring a number and size of particles and/or aerosols in ambient air, a sensor for measuring a concentration of carbon dioxide (CO2); an ambient air temperature sensor, an ambient air relative humidity sensor for the ambient air; and a barometric pressure sensor.

The present invention relates to the field of monitoring the state of health of individuals.

To avoid contamination or to limit the spread of an epidemic, the appearance of a disease must be detected very early. This is particularly true for a respiratory disease, which can spread very quickly, carried by aerosols emitted by a patient, particularly when speaking or sneezing.

It is however difficult for the patient to self-diagnose at the earliest stages of their illness. In addition, many people, especially older people, live alone. Also, a retirement home does not always have sufficient staff for permanent monitoring of residents, particularly at night.

The aim of the invention is to propose a device for the early detection of the occurrence and progression of a respiratory disease in an individual.

According to a first object of the invention, a device for determining the occurrence of a respiratory infection in an individual comprises, in order to collect information locally:
an infrared sensor or camera;
a microphone;
a sensor for measuring a number and size of particles and/or aerosols in ambient air;
a sensor for measuring a concentration of carbon dioxide (CO₂);
an ambient air temperature sensor;
a relative humidity sensor for the ambient air; and
a barometric pressure sensor;
   and, preferably:
a radar or lidar which makes it possible to measure the number of people present and the distance between people; and/or
a sensor for measuring a concentration of volatile organic compounds, in particular ethanol, dihydrogen (H₂) or total volatile organic compounds (TCOV); and/or
an air flow sensor, in order to quantify a renewal of indoor air, inside a room in which the device is installed; and/or,
an ultraviolet rate sensor, ultraviolet having an impact on the survival of a virus in the air.

Such a device preferably comprises a geographic position sensor, preferably by satellite. It may also include processing means for the information collected.

It advantageously includes non-wired means for transmitting the collected information, raw or processed, to a remote server, preferably to a server in a cloud. It may include memory means for transmitting the collected information in a delayed manner.

A second object of the invention relates to a monitoring system which comprises at least one device according to the invention, a remote server for processing the collected information, and non-wired means for transmitting the information processed by the remote server, either to the individual or to another person, said remote server preferably comprising an artificial intelligence algorithm making it possible to recognize a cough.

Advantageously, such a system comprises at least three devices according to the invention.

A third object of the invention relates to a method for determining an occurrence of a pulmonary infection which comprises the use of a system according to the invention, and a measurement of a concentration of droplets having a diameter between one tenth of a micrometre (0.1 μm) and 150 micrometres (150 μm), and, preferably, for droplets whose diameter is less than one micrometre (1 μm), droplets whose diameter is greater than one micrometre (1 μm) and droplets with a diameter greater than one hundred micrometres (100 μm).

A fourth object of the invention relates to a method for determining a geographical evolution of a pulmonary infection, using data collected by a system comprising at least three devices according to the invention.

Several embodiments of the invention will be described hereafter, by way of non-limiting examples, with reference to the appended drawings, in which:

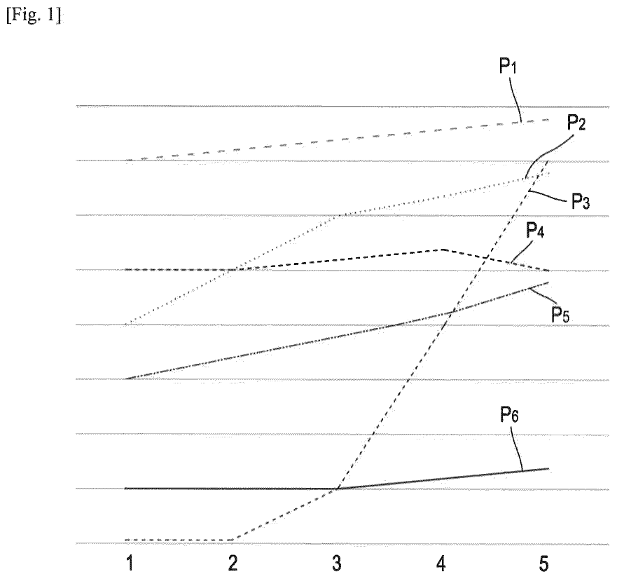

FIG. 1 is an illustration of an example of the evolution of different symptomatic parameters during the first five days of contamination by a respiratory infection; and

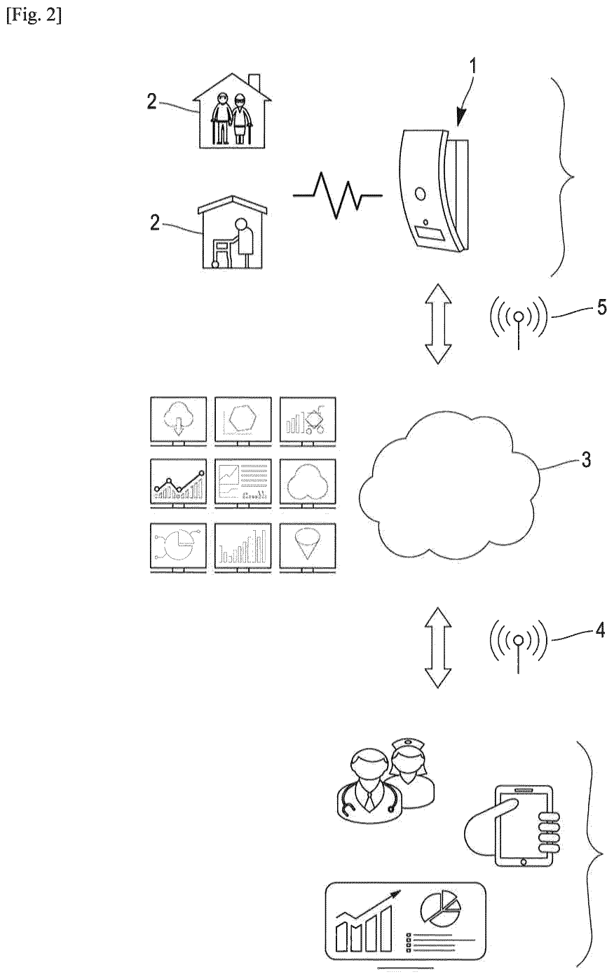

FIG. 2 illustrates schematically a system and a method according to the invention, for determining the occurrence of a respiratory infection.

FIG. 1 represents, by way of example, curves of which each illustrates the evolution of a respective parameter P1-P6 during the first five days of a respiratory infection in an individual. In the example shown:
a first P1 curve illustrates a variation in body temperature of the individual;
a second curve P2 illustrates a variation in a level of carbon dioxide (CO₂) in a room where the individual is staying;
a third curve P3 illustrates a variation in coughing, for example in the frequency of coughing, in the individual;
a fourth curve P4 illustrates a variation in air temperature in a room where the individual is staying;
a fifth curve P5 illustrates a variation in aerosol level in a room where the individual is staying; and
a fifth curve P5 illustrates a variation in humidity in a room where the individual is staying.

It can be seen from these curves that it is possible to evaluate, very early on, the occurrence of a respiratory infection based on these parameters. Advantageously, a device 1 (see FIG. 2) is proposed for detecting the occurrence of such an infection, in particular based on one or more of the parameters.

The device 1 is a non-contact multiparametric sensor combining measurements of physiological symptoms and measurements characterizing the environment. It is used to analyse a closed room 2 in order to assess the risk of respiratory infections. This room can be a room in a residence for elderly or dependent people, or a living room in a home.

The device is used in conjunction with an artificial intelligence solution based in a remote server, in the cloud 3, which generates an infection risk index and 4 provides alerts as well as recommendations for corrective actions.

This hardware and cloud solution provides key information to families, a proxy or a caregiver, providing for example insights into the living conditions of elderly loved ones, so they can make adjustments and act more quickly in the event of an alert. It helps older people improve their health and their safety at home thanks to the early detection of respiratory infection risks. It is also an interesting solution for retirement homes, where it can prevent the spread of respiratory infections through early detection of risks in indoor spaces.

The measuring device includes the following sensors to monitor parameters corresponding to physiological symptoms:
an infrared sensor or camera which can measure a temperature remotely, notably in particular the temperature of the human body remotely, but not only;
a microphone supplemented by processing means implementing an artificial intelligence algorithm making it possible to recognize a cough; and
a radar or lidar which makes it possible to measure the number of people present and the distance between people.

It appears that measuring body temperature associated with coughing can identify a respiratory infection in more than 90% of cases.

The measuring device also includes the following sensors to monitor environmental parameters:
a sensor for measuring the number and size of particles and/or aerosols in the air;
a sensor for measuring a concentration of carbon dioxide (CO₂);
a sensor for measuring a concentration of volatile organic compounds, in particular ethanol, dihydrogen (H₂) or total volatile organic compounds (TCOV);
an ambient air temperature sensor;
a relative humidity sensor for the ambient air; and
a barometric pressure sensor.

The device may also include a satellite geographic position sensor. Such a sensor is particularly advantageous if the premises it equips is a mobile enclosure, for example a transport vehicle. This vehicle could be a school bus or a cruise ship, for example.

The device 1 further comprises remote transmission means 5, to transmit the information collected in the room, to the remote server, in the cloud 3. Preferably, the means of transmission are non-wired means, and may, for example, include cellular, Bluetooth and/or Wi-Fi connection means. The absence of a wired connection makes it possible to maintain a flexible installation, in particular if the configuration of the room is changed or if a monitored person moves.

The device can be designed to send the collected information in real time and/or to include a memory for storing this information and sending it in a delayed manner.

The transmission means can also allow the device 1 to receive data, in particular configuration data and/or an update of software hosted in the processing means.

Measuring the concentration of aerosols, as well as their sizes, makes it possible to confirm the presence of a respiratory infection in at least one person present in the room. It has been identified that an infected person generates more aerosols and droplets when breathing or talking or coughing as compared to a healthy person.

In addition, the size of droplets and aerosols makes it possible to identify their origin. Thus, depending on the areas:
the film of bronchiolar fluid, during respiration, produces droplets whose diameter is less than one micrometre (1 μm);
the fluid which coats the respiratory tract produces, in laryngeal mode, during coughing and speaking, droplets whose diameter is greater than one micrometre (1 μm); and
the droplets coming from the cavity during coughing and speaking have a diameter greater than one hundred micrometres (100 μm).

Viruses and bacteria are preferentially present in one or the other of these areas (bronchioles, respiratory tract or oral cavity). Monitoring the concentration and measuring the size of particles, droplets or aerosols having a diameter comprised between one-tenth of a micrometre (0.1 μm) and 150 micrometres (150 μm) is therefore relevant and makes it possible to differentiate between types and sources of infection.

The sensor that is mainly used for measuring human body temperature is the infrared sensor. Since human body temperature can vary depending on several parameters external to the human body—including notably ambient temperature, ambient humidity, lighting level or ambient ultraviolet index—relying only on the parameter measured by this sensor can lead to an incorrect measured value.

To correct the measurement of human body temperature by infrared and make it more accurate and precise, this measurement is correlated with other measurements from other sensors present in the product. The infrared measurement will be corrected according to the parameters measured by the other sensors. The measurements and information provided by the different sensors at the same time and in the same place make it possible to ensure a measurement of the temperature of the human body is more reliable.

For each of the sensors, a measured parameter can be used raw or corrected according to other measured parameters.

The device is designed to operate autonomously and automatically. For this, the device 1, using its multiple sensors, allows a self-checking function. Thus, the infrared sensor has known and specified limits of use, in particular in intervals of ambient temperature, ambient humidity, atmospheric pressure, light incidence or ultraviolet level. In order to guarantee optimal operation of the device 1, in particular to ensure that the measurements made by the infrared sensor are reliable, the measurements of the various other sensors are used to verify that the infrared sensor operates within the correct intervals.

The device 1 can in particular be used for the safety of individuals, in particular elderly people living alone.

The device measures the temperature of an individual and thus determines their presence in the room. The device can also determine whether the person is immobile or not. In the event of prolonged immobility, and in the event of a notable reduction in the sound level in the room, the device can, for example, deduce that the individual has suffered a collapse or has fallen and trigger an alarm and/or contact a correspondent.

As each device is connected to the cloud, the data collected by each device can be correlated with each other in order to provide information on a given geographic area. For example, several devices providing an increase in CO₂ thus make it possible to provide information on this pollution and a direction of movement of this pollution. The same can be true for measured volatile compounds.

This provision of information resulting from a combination of one or more of the parameters measured by the device may have uses other than monitoring the state of health of an individual. For example, the device can make it possible to measure vibrations such as those produced by an earthquake; it can make it possible, if the mesh is sufficient, to determine its amplitude and epicentre.

It is also possible to measure and monitor the evolution, in a given area:
of a sound level;
of a level of pressure, temperature and/or humidity;
of a level of brightness and/or sunshine.

Of course, the invention is not limited to the examples which have just been described. On the contrary, the invention is defined by the claims which follow.

It will indeed appear to those skilled in the art that various modifications can be made to the embodiments described above, in light of the teaching which has just been disclosed to them.

Thus, the device can in particular include an infrared sensor designed to measure a temperature in several angular sectors, so that it can simultaneously and individually measure the temperature of several people and/or objects in the room, so that it can differentiate an object from a person, or two people from each other.

A sensor and a system according to the invention fulfil three main functions:
a measurement of physiological symptoms, such as body temperature, detection of coughs and sneezes;
a measurement of transmissibility in the air, in particular by the presence of aerosols, droplets and air flow; and
a measurement of the potential survival of a virus in the air, in particular as a function of ultraviolet rays, temperature and humidity of the air.

A device and a system according to the invention have numerous advantages:
It has several sensors, which allows simultaneous physical measurements and allows correlation analyses between different parameters, for example between pollution by aerosols and/or volatile compounds and body temperature. This also allows the use of an artificial intelligence algorithm to find complex correlations between different parameters.
The connection to a remote server, in particular a server in the cloud, allows remote analysis of the collected information by the device. Thus, the use of the device can be modified by merely changing a computer program in the remote server, services provided from the collected information may be different depending on the use made thereof.

Of course, such a device and such a system can be used in classrooms or in offices.

A device and a system according to the invention are not limited to the monitoring of an airborne disease, but also of other diseases transmissible through air, for example gastroenteritis.

What is claimed is:

1-9. (canceled)

10. System for determining an occurrence of an airborne disease, in particular a respiratory infection, in an individual present in a closed room, comprising:
at least one contactless multiparameter sensor, placed in a room, combining the measurement of physiological symptoms and measurements characterising the environment,
a remote server for processing the collected information, and
non-wired means for transmitting the information processed by the remote server, either to the individual or to another person, said remote server preferably comprising an artificial intelligence algorithm making it possible to recognize a cough,
wherein said system is configured for establishing three functions of:
a measurement of physiological symptoms,
a measurement of transmissibility in the air, and
a measurement of the potential survival of a virus in the air.

11. System according to claim 10, wherein the function of measuring physiological symptoms comprises the measurement of the body temperature of the individual, the detection of coughs and sneezes.

12. System according to claim 10, wherein the function of measuring the transmissibility in the air comprises the detection of the presence of aerosols, droplets and air flow.

13. System according to claim 10, wherein the function of measuring the potential survival of a virus in the air comprises the detection of ultraviolet rays, of temperature and of humidity of the air.

14. System according to claim 10, wherein said measurement of physiological symptoms are performed by the following sensors:
an infrared sensor or camera adapted for measuring a temperature remotely, in particular the temperature of the individual;
a microphone adapted for being connected to processing means implementing an artificial intelligence algorithm making it possible to recognize a cough; and
a radar or lidar adapted for measuring a number of people present in the room and the distance between people.

15. System according to claim 14, wherein said sensor or infrared camera is adapted for measuring a temperature in several angular sectors.

16. System according to claim 10, wherein said measurement of environmental parameters are performed by the following sensors:
a sensor adapted for measuring a number and size of particles and/or aerosols in the air;
a sensor adapted for measuring a concentration of carbon dioxide (CO₂);
a sensor adapted for measuring a concentration of volatile organic compounds, in particular ethanol, dihydrogen (H₂) or total volatile organic compounds (COVT);
an ambient air temperature sensor;
a relative humidity sensor for the ambient air; and
a barometric pressure sensor.

17. System according to claim 10, wherein said multiparameter sensor further comprises a geographic position sensor.

18. System according to claim 10, wherein the multiparametric sensor comprises:
an infrared sensor or camera;
a microphone;
a sensor for measuring a number and size of particles and/or aerosols in ambient air;
a sensor for measuring a concentration of carbon dioxide (CO₂);
an ambient air temperature sensor;
a relative humidity sensor for the ambient air; and
a barometric pressure sensor;

19. System according to claim 18, wherein the multiparametric sensor further comprises:
a radar or lidar which makes it possible to measure the number of people present and the distance between people; and/or
a sensor for measuring a concentration of volatile organic compounds, in particular ethanol, dihydrogen (H₂) or total volatile organic compounds (COVT); and/or
an air flow sensor; and/or
an ultraviolet rate sensor.

20. System according to claim 10, wherein the at least one multiparameter sensor further comprises processing means for the collected information.

21. System according to claim 10, wherein the at least one multiparameter sensor further comprises non-wired means adapted for transmitting the collected information, raw or processed, to the remote server.

22. System according to claim 10, wherein the multiparameter sensor comprises memory means for transmitting the collected information in a delayed manner.

23. System according to claim 10, further comprising at least three multiparameter sensors.

24. Method for determining an occurrence of an airborne disease, in particular a respiratory infection, with the system according to claim 10, comprising one or several steps from among:
measuring a concentration of droplets having a diameter between one tenth of a micrometre (0.1 μm) and 150 micrometres (150 μm), and, preferably, for droplets whose diameter is less than one micrometre (1 μm), droplets whose diameter is greater than one micrometre (1 μm) and droplets with a diameter greater than one hundred micrometres (100 μm),
determine the presence and/or immobility of an individual in the room by measuring their temperature and/or the sound level in the room,
determining the temperature of the human body of an individual present in the room by means of said sensor or of an infrared camera, and correlate the temperature measurement to the other measurements of the other sensors of said system,
performing a self-checking measurement of the infrared sensor by means of the measurements of the other sensors,
correcting a measured parameter by means of other measured parameters,
correlating the collected data from each of the multiparameter sensors by the remote server,
performing simultaneous physical measurements and correlation analyses between the different measured parameters,
modifying the use of at least one multiparameter sensor by changing the program of said remote server.

25. Method for determining a geographical evolution of an airborne disease, in particular a respiratory infection, with the system according to claim 10, wherein the method uses data collected by at least three multiparameter sensors.
